(19) United States
(12) Patent Application Publication
(10) Pub. No.: US 2025/0370162 A1
(43) Pub. Date: Dec. 4, 2025

(54) METHOD AND APPARATUS FOR EVALUATING CO2 STORAGE POTENTIAL UNDER HETEROGENEOUS GEOLOGICAL CONDITION

(71) Applicant: China University of Petroleum-Beijing, Beijing City (CN)

(72) Inventors: Leilei YANG, Beijing City (CN); Yi LIU, Beijing City (CN); Zhenhua RUI, Beijing City (CN); Ting HU, Beijing City (CN); Xiaowei LI, Beijing City (CN); Ziyang SONG, Beijing City (CN); Shu XU, Beijing City (CN); Wenhao YANG, Beijing City (CN); Dawei WANG, Beijing City (CN); Hui WANG, Beijing City (CN); Dongdong LI, Beijing City (CN); Jinlu ZHAO, Beijing City (CN); Nanxin LUO, Beijing City (CN); Yongyue XU, Beijing City (CN)

(73) Assignee: China University of Petroleum-Beijing, Beijing City (CN)

(21) Appl. No.: 19/067,135

(22) Filed: Feb. 28, 2025

(30) Foreign Application Priority Data
May 30, 2024 (CN) 202410691080.3

(51) Int. Cl. G01V 20/00; E21B 41/00

(52) U.S. Cl. CPC G01V 20/00; E21B 41/0064; E21B 2200/20

(57) ABSTRACT

There is a method for evaluating CO₂ storage potential under a heterogeneous geological condition. The method has the steps of acquiring a plurality pieces of geological data at different depths for any location in a study area; performing heterogeneity modeling according to the plurality pieces of geological data and CO₂ injection conditions to obtain one-dimensional grid models respectively corresponding to a plurality of the different depths; obtaining, based on the one-dimensional grid models, CO₂ storage capacities at the plurality of different depths through simulation; integrating the CO₂ storage capacities and fitting the plurality pieces of geological data to obtain a main control factor affecting the CO₂ storage capacity; and performing analysis based on the main control factor and determining a formation affected by the main control factor as a formation with CO₂ storage potential. There is also an apparatus and a computer device

### CROSS-REFERENCE TO RELATED APPLICATION

The present disclosure claims priority to Chinese Patent Application No. 202410691080.3, filed on May 30, 2024, which is hereby incorporated by reference in its entirety.

### FIELD OF THE DISCLOSURE

The embodiments of the present disclosure relate to the field of carbon storage, and in particular to a method and apparatus for evaluating CO₂ storage potential under a heterogeneous geological condition.

### DESCRIPTION OF THE RELATED ART

CO₂ storage is a key technology in addressing climate change, which reduces the concentration of CO₂ in the atmosphere by permanently storing CO₂ in formations, thereby responding to current policies and achieving carbon neutrality. Under the conditions of different heterogeneous formations and different factors (including temperature, pressure, geological structure, mineral composition, etc.), the feasibility and efficiency of carbon storage are significantly impacted. Therefore, accurate assessment of CO₂ storage potential is crucial to guide the implementation of carbon storage projects.

At present, carbon storage capacity calculation methods in the related art are often based on average properties of formations. The calculation is macroscopic and the complex heterogeneity within the formations is ignored. This leads to increased uncertainty in calculation results and reduced accuracy in evaluating CO₂ storage potential.

Therefore, there is an urgent need for a method for evaluating CO₂ storage potential under a heterogeneous geological condition that can improve the accuracy of CO₂ storage potential evaluation.

### SUMMARY OF THE DISCLOSURE

An object of the embodiments of the present disclosure is to provide a method and apparatus for evaluating CO₂ storage potential under a heterogeneous geological condition, to improve the accuracy of CO₂ storage potential evaluation.

In order to implement the above object, in one aspect, the embodiments of the present disclosure provides a method for evaluating CO₂ storage potential under a heterogeneous geological condition, the method including:
acquiring a plurality pieces of geological data at different depths for any location in a study area;
performing heterogeneity modeling according to the plurality pieces of geological data at each of the depths and CO₂ injection conditions, to obtain one-dimensional grid models respectively corresponding to a plurality of the different depths, and the CO₂ injection conditions of the one-dimensional grid models respectively corresponding to the plurality of different depths are the same;
obtaining, according to the one-dimensional grid models respectively corresponding to the plurality of different depths, CO₂ storage capacities at the plurality of different depths through simulation;
integrating the CO₂ storage capacities at the plurality of different depths and fitting the plurality pieces of geological data, to obtain a main control factor that affects the CO₂ storage capacity; and
analyzing formations at the different depths in the study area based on the main control factor, and determining a formation affected by the main control factor as a formation with CO₂ storage potential.

Exemplarily, the plurality pieces of geological data include logging data and formation water data.

Exemplarily, the performing heterogeneity modeling according to the plurality pieces of geological data at each of the depths and the CO₂ injection conditions, to obtain one-dimensional grid models respectively corresponding to the plurality of the different depths further includes:
preprocessing the plurality pieces of geological data at each of the depths to obtain a plurality pieces of preprocessed geological data at each of the depths, and
simulating formation characteristics at each of the depths using a simulation tool, according to the plurality pieces of preprocessed geological data at each of the depths and the CO₂ injection conditions, to obtain one-dimensional grid models respectively corresponding the plurality of different depths.

Exemplarily, the integrating the CO₂ storage capacities at the plurality of different depths and fitting the plurality pieces of geological data, to obtain the main control factor that affects the CO₂ storage capacity further includes:
integrating the CO₂ storage capacities at the plurality of different depths and the same geological data at the plurality of different depths, to obtain correlations between the CO₂ storage capacity and each geological data in the study area, and
analyzing, according to the correlations, a degree of affection of each geological data on the CO₂ storage capacity to obtain the main control factor that affects the CO₂ storage capacity.

Exemplarily, the integrating the CO₂ storage capacities at the plurality of different depths and the same geological data at the plurality of different depths, to obtain the correlations between the CO₂ storage capacity and each geological data in the study area further includes:
constructing scatter plots corresponding to the geological data using the CO₂ storage capacities at the plurality of different depths and the same geological data at the plurality of different depths as a plurality of groups of ordinate values and abscissa values, respectively, and
obtaining, based on the scatter plots corresponding to each geological data, correlations between the CO₂ storage capacity and each geological data in the study area.

Exemplarily, the analyzing, according to the correlations, the degree of affection of each geological data on the CO₂ storage capacity, to obtain the main control factor that affects the CO₂ storage capacity further includes:
obtaining, according to the scatter plots corresponding to each geological data, linear regression models corresponding to each geological data,
calculating, according to the linear regression models, goodness-of-fit values corresponding to each geological data,
setting geological data corresponding to a goodness-of-fit value greater than a set value or a maximum value among all goodness-of-fit values as target geological data, and
obtaining, according to the linear regression model corresponding to the target geological data, the main control factor that affects the CO₂ storage capacity.

Exemplarily, the obtaining, according to the linear regression model corresponding to the target geological data, the main control factor that affects the CO₂ storage capacity further includes:
obtaining, according to the linear regression model corresponding to the target geological data, a proportional relation between the target geological data and the CO₂ storage capacity, and
setting the target geological data and the proportional relation between the target geological data and the CO₂ storage capacity as the main control factor that affects the CO₂ storage capacity.

In another aspect, the embodiments of the present disclosure provide an apparatus for evaluating CO₂ storage potential under a heterogeneous geological condition, including:
an acquisition module configured to acquire a plurality pieces of geological data at different depths for any location in a study area;
a modeling module configured to perform heterogeneity modeling according to the plurality pieces of geological data at each of the depths and CO₂ injection conditions, to obtain one-dimensional grid models respectively corresponding to a plurality of the different depths, and the CO₂ injection conditions of the one-dimensional grid models respectively corresponding to the plurality of different depths are the same;
an operation module configured to obtain, based on the one-dimensional grid models respectively corresponding to the plurality of different depths, CO₂ storage capacities at the plurality of different depths through simulation;
a main control factor determination module configured to integrate the CO₂ storage capacities at the plurality of different depths and fitting the plurality pieces of geological data, to obtain a main control factor that affects the CO₂ storage capacity; and
an analysis module configured to analyze formations at the different depths in the study area based on the main control factor, and determine a formation affected by the main control factor as a formation with CO₂ storage potential.

In yet another aspect, the embodiments of the present disclosure further provide a computer device, including: a memory; a processor; and a computer program stored in the memory, in which when being executed by the processor, the computer program implements an instruction according to any one of the above methods.

In yet another aspect, the embodiments of the present disclosure further provide a computer-readable storage medium storing a computer program, in which when being executed by the processor of a computer device, the computer program implements an instruction according to any one of the above methods.

It can be seen from the technical solutions provided in the above embodiments of the present disclosure that, according to the method of the embodiments of the present disclosure, heterogeneity modeling is performed based on a plurality pieces of geological data at a plurality of different depths for any location in a study area, and thus one-dimensional grid models respectively corresponding to the plurality of the different depths can be obtained. Boundary conditions of the plurality of one-dimensional grid models are limited to the same CO₂ injection condition, and then a CO₂ storage capacity is simulated to obtain CO₂ storage capacities at the plurality of different depths. Further, the CO₂ storage capacities at the plurality of different depths are integrated, and the plurality pieces of geological data are fitted to obtain a main control factor. Based on the main control factor, the entire study area can be analyzed to obtain a formation with CO₂ storage potential. In this way, the carbon storage capacity can be accurately calculated using advanced heterogeneity modeling technology and simulation calculation methods.

In order to make the above and other objects, features and advantages of the present disclosure more obvious and easy to understand, embodiments are specifically exemplified below and described in detail with reference to the accompanying drawings.

### BRIEF DESCRIPTION OF DRAWINGS

In order to describe the embodiments of the present disclosure or technical solutions in the related art more clearly, the drawings required for the description of the embodiments or the related art will be briefly described below. Obviously, the drawings in the following description are merely some embodiments of the present disclosure. A person skilled in the art can obtain other drawings based on these drawings without making inventive efforts.

FIG. 1 shows a schematic flowchart of a method for evaluating CO₂ storage potential under a heterogeneous geological condition according to an embodiment of the present disclosure.

FIG. 2 shows a schematic flowchart of a method for dividing different depths according to the embodiment of the present disclosure.

FIG. 3 shows a schematic flowchart of obtaining one-dimensional grid models respectively corresponding to a plurality of the different depths according to the embodiment of the present disclosure.

FIG. 4 is a schematic flowchart of obtaining a main control factor that affects CO₂ storage capacity according to the embodiment of the present disclosure.

FIG. 5 shows a schematic flowchart of obtaining a correlation between the CO₂ storage capacity and each geological data in a study area according to the embodiment of the present disclosure.

FIG. 6 shows a schematic flowchart of analyzing a degree of affection of each geological data on the CO₂ capacity to obtain a main control factor that affects the CO₂ storage capacities according to the embodiment of the present disclosure.

FIG. 7 shows a schematic flowchart of obtaining, according to a linear regression model corresponding to target geological data, a main control factor that affects the CO₂ storage capacity, according to the embodiment of the present disclosure.

FIG. 8 shows a scatter plot and a linear regression model corresponding to the feldspar mineral content according to the embodiment of the present disclosure.

FIG. 9 shows a scatter plot and a linear regression model corresponding to the clay mineral content according to the embodiment of the present disclosure.

FIG. 10 shows a scatter plot and a linear regression model corresponding to the quartz content according to the embodiment of the present disclosure.

FIG. 11 shows a scatter plot and a linear regression model corresponding to the carbonate mineral content according to the embodiment of the present disclosure.

FIG. 12 shows a schematic diagram of the distribution of the feldspar mineral content and the CO₂ storage capacities under the different depths according to the embodiment of the present disclosure.

FIG. 13 shows a schematic diagram of the distribution of the clay mineral content and the CO₂ storage capacities under the different depths according to the embodiment of the present disclosure.

FIG. 14 shows a schematic diagram of a module structure of an apparatus for evaluating CO₂ storage potential under a heterogeneous geological condition according to the embodiment of the present disclosure.

FIG. 15 shows a schematic diagram of a structure of a computer device according to the embodiment of the present disclosure.

### REFERENCE SIGNS LIST

100: acquisition module
200: modeling module
300: operation module
400: main control factor determination module
500: analysis module
1502: computer device
1504: processor
1506: memory
1508: drive mechanism
1510: input and output module
1512: input device
1514: output device
1516: display device
1518: graphical user interface
1520: network interface
1522: communication link
1524: communication bus

### DETAILED DESCRIPTION OF THE DISCLOSURE

The technical solutions of the embodiments of the present disclosure will be clearly and completely described below with reference to the drawings in the embodiments of the present disclosure. Obviously, the described embodiments are some, not all, of the embodiments of the present disclosure. All other embodiments obtained by a person skilled in the art according to the embodiments of the present disclosure without making inventive efforts are within the scope of protection of the embodiments of the present disclosure.

At present, carbon storage capacity calculation methods in the related art are often based on average properties of formations. The calculation is macroscopic and the complex heterogeneity within the formations is ignored. This leads to increased uncertainty in calculation results and reduced accuracy in evaluating CO₂ storage potential.

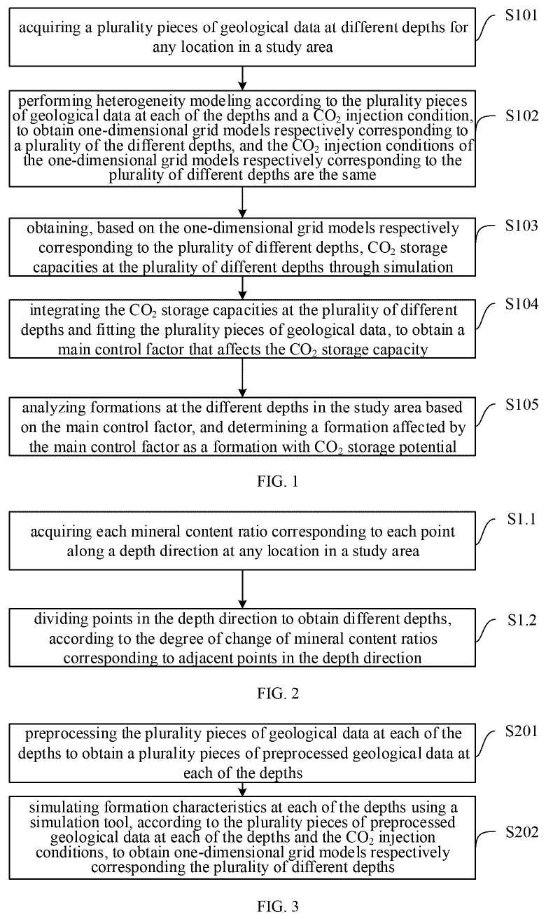

In order to solve the above problems, the embodiments of the present disclosure provide a method for evaluating CO₂ storage potential under a heterogeneous geological condition. FIG. 1 is a schematic flowchart of the method for evaluating CO₂ storage potential under a heterogeneous geological condition according to an embodiment of the present disclosure. The present disclosure provides method operation steps as described in the embodiments or flowcharts, but may include more or fewer operation steps according to conventional or non-inventive efforts. The step order listed in the embodiments is merely one of a plurality of step execution orders and does not represent the only execution order. When executed in an actual system or device, the methods shown in the embodiments or drawings may be executed sequentially or in parallel.

It should be noted that the terms “first”, “second”, etc. in the disclosure and claims of the embodiments of the present disclosure and the above drawings are used to distinguish similar objects, and are not necessarily used to describe a specific order or sequence. It should be understood that the data used in this way can be interchanged where appropriate such that the embodiments of the present disclosure described herein can be implemented in an order other than those illustrated or described herein. In addition, the terms “include” and “comprise” and any variations thereof are intended to cover non-exclusive inclusions. For example, processes, methods, apparatus, products, or device that includes a series of steps or units is not necessarily limited to those steps or units that are clearly listed, but may include other steps or units that are not clearly listed or inherent to these processes, methods, products, or device.

With reference to FIG. 1, an embodiment of the present disclosure provides a method for evaluating CO₂ storage potential under a heterogeneous geological condition, including:
S101: acquiring a plurality pieces of geological data at different depths for any location in a study area;
S102: performing heterogeneity modeling according to the plurality pieces of geological data at each of the depths and a CO₂ injection condition, to obtain one-dimensional grid models respectively corresponding to a plurality of the different depths, and the CO₂ injection conditions of the one-dimensional grid models respectively corresponding to the plurality of different depths are the same;
S103: obtaining, based on the one-dimensional grid models respectively corresponding to the plurality of different depths, CO₂ storage capacities at the plurality of different depths through simulation;
S104: integrating the CO₂ storage capacities at the plurality of different depths and fitting the plurality pieces of geological data, to obtain a main control factor that affects the CO₂ storage capacity; and
S105: analyzing formations at the different depths in the study area based on the main control factor, and determining a formation affected by the main control factor as a formation with CO₂ storage potential.

In this embodiment, the plurality pieces of geological data include logging data and formation water data. The logging data includes temperature, pressure, porosity, permeability, mineral type, mineral content, etc., and the formation water data includes the concentration of different ions in formation water, pH (pondus hydrogenii) value of formation water, etc. In order to obtain a plurality pieces of geological data at different depths for any location, it is necessary to drill a well for exploration at the location if there is no existing well at the location, and the data of an existing well can be directly used if there is an existing well at the location.

In the embodiments of the present disclosure, different depths can be divided according to requirements, and there are a plurality pieces of geological data corresponding to each depth. When dividing different depths, different depths can be divided by a set distance. For example, every 10 meters is used as a set distance to obtain a plurality pieces of geological data at different depths between 2600 meters and 3300 meters. The set distance can be set according to actual conditions of a study area. For example, a plurality pieces of geological data at 2600 meters are acquired, a plurality pieces of geological data at 2610 meters are acquired, a plurality pieces of geological data at 2620 meters are acquired, . . . , and a plurality pieces of geological data at 3300 meters are acquired.

When dividing different depths, different depths can also be divided according to varying distances. For example, if the depth is between 2,600 meters and 3,300 meters, 2,600 meters is divided into one depth, 2,605 meters is divided into one depth, and 2,612 meters is divided into one depth, and so on. The difference between adjacent depths is a varying distance. With reference to FIG. 2, a method for dividing different depths may include:
S1.1: acquiring each mineral content ratio corresponding to each point along a depth direction at any location in a study area; and
S1.2: dividing points in the depth direction to obtain different depths, according to the degree of change of mineral content ratios corresponding to adjacent points in the depth direction.

Specifically, mineral types in one study area may include carbonate, quartz, feldspar, etc. In general, mineral types at any location in a study area are the same along a depth direction, except that the content of different minerals may be different. At the time of logging, an even measurement is performed using logging equipment along a depth direction, and thus mineral content ratios at several points evenly distributed along the depth direction can be obtained. The points are determined by an accuracy value of a measuring equipment. For example, a measuring equipment measures once every 1 meter, that is, an interval between any two adjacent points is 1 meter, and mineral content ratios corresponding to the points between 2600 meters and 3300 meters can be obtained.

Assuming that mineral components in a study area include carbonate, quartz, and feldspar, and point A, point B, point C, etc. are sequentially designed in a depth direction. The content ratio of carbonate, quartz, and feldspar at the point Ais 1:1:1, the content ratio of carbonate, quartz, and feldspar at the point B is 1:2:2, and the content ratio of carbonate, quartz, and feldspar at the point Cis 1:2:1, and so on. The degree of change of mineral content ratios corresponding to any two adjacent points is further analyzed. The degree of change of mineral content ratios corresponding to two adjacent points refers to the degree of change of the lower point relative to the upper point in the depth direction, for example, the degree of change of the mineral content ratio at the point B relative to that at the point A, and the degree of change of the mineral content ratio at the point C relative to that at the point B. Specifically, an overall change rate of the mineral content ratio at each point can be calculated to represent the degree of change, The overall change rate is calculated by calculating a change rate of each mineral content, and summing the change rates of each mineral to obtain the overall change rate. When the overall change rate is greater than a set change rate, two adjacent points are classified into different depth ranges. When the overall change rate is less than or equal to the set change rate, two adjacent points are classified into the same depth range. Different depth ranges correspond to different depths, and the same depth range corresponds to the same depth. In this way, a plurality of different depth ranges are obtained. For each depth range, any point in the depth range is taken as a representation point, and different depths are obtained through representation points.

For example, the point A and the point B are two adjacent points, a carbonate change rate is 0, a quartz change rate is (2−1)/1=100%, a feldspar change rate is 100%, and an overall change rate is 200%. If the overall change rate is greater than a set change rate, the point A and the point B are at different depths. Assuming that the point A is at a depth of 2,604 meters and the point B is at 2,605 meters, the point A and the point B are in different depth ranges, the point A may in a depth range of 2,600 meters to 2,604 meters, and the point B may in a depth range of 2,605 meters to 2,612 meters. Different depth ranges correspond to different depths, and each depth range can be represented by any point in the depth range, such as a left endpoint. The depth range of 2,600 meters to 2,604 meters is represented by 2,600 meters, and the depth range of 2,605 meters to 2,612 meters is represented by 2,605 meters. This division results in two different depths of 2,600 meters and 2,605 meters.

The TOUGHREACT simulation tool is used for heterogeneity modeling. TOUGHREACT is a multiphase fluid and reactive chemistry simulation tool. When performing heterogeneity modeling, it is necessary to establish a one-dimensional grid model for each depth to obtain one-dimensional grid models respectively corresponding to the plurality of different depths. In order to ensure that the one-dimensional grid models respectively corresponding different depths are operated under the same conditions and to ensure the accuracy of subsequent simulation results, a CO₂ injection conditions of the one-dimensional grid models respectively corresponding to the plurality of different depths must be the same.

Specifically, with reference to FIG. 3, performing heterogeneity modeling based on the plurality pieces of geological data at each of the depths and the CO₂ injection condition, to obtain one-dimensional grid models respectively corresponding to the plurality of the different depths further includes:
S201: preprocessing the plurality pieces of geological data at each of the depths to obtain a plurality pieces of preprocessed geological data at each of the depths; and
S202: simulating formation characteristics at each of the depths using a simulation tool, according to the plurality pieces of preprocessed geological data at each of the depths and the CO₂ injection conditions, to obtain one-dimensional grid models respectively corresponding the plurality of different depths.

Preprocessing is to ensure the integrity and consistency of the plurality pieces of geological data. Specifically, missing values can be processed through interpolation methods. The CO₂ injection condition includes a CO₂ injection location, a CO₂ injection rate, CO₂ injection time, etc. The CO₂ injection condition is used as a boundary condition of the one-dimensional grid models. According to the preprocessed plurality pieces of geological data at each depth, a refined one-dimensional grid model is established and accurately assigned to reflect the real physical and chemical properties of formations at different depths. In this way, one-dimensional grid models respectively correspond to the plurality of different depths can be obtained.

After obtaining the one-dimensional grid models respectively correspond to the plurality of different depths, the TOUGHREACT simulation tool can be executed to simulate the one-dimensional grid models respectively corresponding to the plurality of different depths, thereby obtaining CO₂ storage capacities at the plurality of different depths, which facilitates the analysis of CO₂ storage effects at different depths under the same injection condition. The CO₂ storage capacities after 100 years, 1,000 years or 10,000 years can be simulated, and the embodiment of the present disclosure does not limit the simulation time.

After obtaining the CO₂ storage capacities at the plurality of different depths, the CO₂ storage capacities can be integrated to obtain the CO₂ storage capacities at different depths for a corresponding location in the study area, and the plurality pieces of geological data can be fitted based on that CO₂ storage capacities. The plurality pieces of geological data need to be fitted one by one. Since the CO₂ storage capacities at the plurality of different depths for the corresponding location are known, and the same geological data at the plurality of different depths is known (such as temperature, porosity or feldspar mineral content, etc.), a main control factor that affects the CO₂ storage capacity can be obtained by fitting. It should be noted that the main control factor includes at least one piece of geological data, and a proportional relation between the geological data and the CO₂ storage capacity (for example, the content of feldspar mineral is proportional to the CO₂ storage capacity).

There is at least one main control factor. The main control factor is the main reason affecting the CO₂ storage capacity in the study area. After obtaining the main control factor, the entire study area can be analyzed based on the main control factor, and a formation with CO₂ storage potential among formations at different depths in the study area can be analyzed and determined according to the main control factor. For example, assuming that the main control factor is the feldspar content, and the feldspar content is proportional to the CO₂ storage capacity, a formation with higher feldspar content in the entire study area can be analyzed and used as the formation with CO₂ storage potential.

According to the method of the embodiment of the present disclosure, heterogeneity modeling is performed based on a plurality pieces of geological data at a plurality of different depths for any location in a study area, and thus one-dimensional grid models respectively corresponding to the different depths can be obtained. Boundary conditions of the plurality of one-dimensional grid models are limited to the same CO₂ injection condition, and then a CO₂ storage capacity is simulated to obtain the CO₂ storage capacities at the plurality of different depths. Further, the CO₂ storage capacities at the plurality of different depths are integrated, and the plurality pieces of geological data are fitted, thereby obtaining a main control factor. Based on the main control factor, the entire study area can be analyzed to obtain a formation with CO₂ storage potential. In this way, the carbon storage capacity can be accurately calculated using advanced heterogeneity modeling technology and simulation calculation methods.

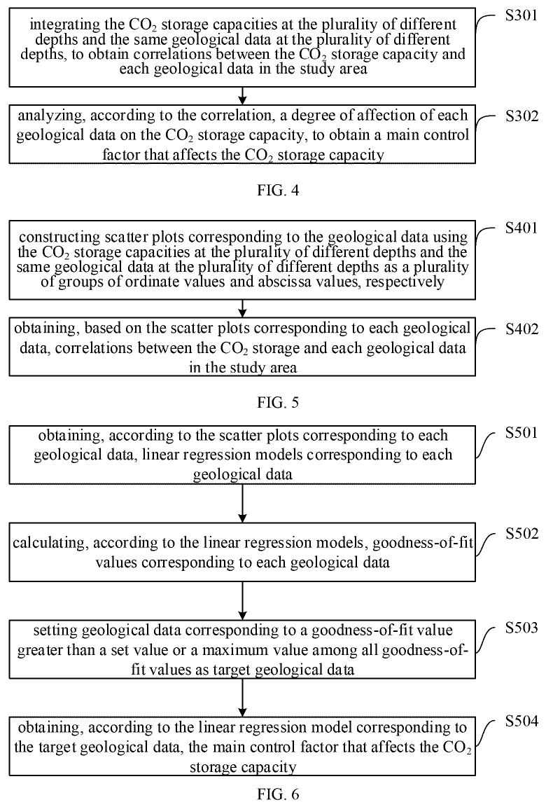

In the embodiment of the present disclosure, with reference to FIG. 4, integrating the CO₂ storage capacities at the plurality of different depths and fitting the plurality pieces of geological data, to obtain the main control factor that affects the CO₂ storage capacity further includes:
S301: integrating the CO₂ storage capacities at the plurality of different depths and the same geological data at the plurality of different depths, to obtain correlations between the CO₂ storage capacity and each geological data in the study area; and
S302: analyzing, according to the correlation, a degree of affection of each geological data on the CO₂ storage capacity, to obtain a main control factor that affects the CO₂ storage capacity.

Specifically, with reference to FIG. 5, integrating the CO₂ storage capacities at the plurality of different depths and the same geological data at the plurality of different depths, to obtain the correlations between the CO₂ storage capacity and each geological data in the study area further includes:
S401: constructing scatter plots corresponding to the geological data using the CO₂ storage capacities at the plurality of different depths and the same geological data at the plurality of different depths as a plurality of groups of ordinate values and abscissa values, respectively; and
S402: obtaining, based on the scatter plots corresponding to each geological data, correlations between the CO₂ storage and each geological data in the study area.

Taking the feldspar mineral content in geological data as an example, at each depth, there is corresponding CO₂ storage capacity and a feldspar mineral content. The CO₂ storage capacity at each depth is used as an ordinate value, and the corresponding feldspar mineral content is used as an abscissa value. Thus, a plurality of groups of ordinate values and abscissa values can be obtained, thereby constructing a scatter plot corresponding to the geological data. Similarly, each geological data corresponds to a scatter plot. Since the scatter plot is obtained based on the CO₂ storage capacity and the geological data, the scatter plots corresponding to each geological data can be used as correlations between the CO₂ storage capacity and each geological data.

In the embodiment of the present disclosure, with reference to FIG. 6, analyzing, according to the correlation, the degree of affection of each geological data on the CO₂ storage capacity, to obtain the main control factor that affects the CO₂ storage capacity further includes:
S501: obtaining, according to the scatter plots corresponding to each geological data, linear regression models corresponding to each geological data;
S502: calculating, according to the linear regression models, goodness-of-fit values corresponding to each geological data;
S503: setting geological data corresponding to a goodness-of-fit value greater than a set value or a maximum value among all goodness-of-fit values as target geological data; and
S504: obtaining, according to the linear regression model corresponding to the target geological data, the main control factor that affects the CO₂ storage capacity.

The scatter plots corresponding to each geological data are fitted to obtain linear regression models. In general, the linear regression model is in the form of a straight line, which represents a linear relation between the geological data and the CO₂ storage capacity. For the linear regression model, the R² value can be calculated as a goodness-of-fit value. Each geological data has a corresponding goodness-of-fit value, and the goodness-of-fit value is judged based on a set value. If there is a goodness-of-fit value greater than the set value, the geological data corresponding to the goodness-of-fit value is target geological data. If there is no goodness-of-fit value greater than the set value, the geological data corresponding to the greatest one of all goodness-of-fit values is used as the target geological data. The set value can be set according to actual conditions, which is not limited by the embodiment of the present disclosure. Thus, at least one target geological data is obtained, and the main control factor that affects the CO₂ storage capacity can be obtained according to the linear regression model corresponding to the target geological data.

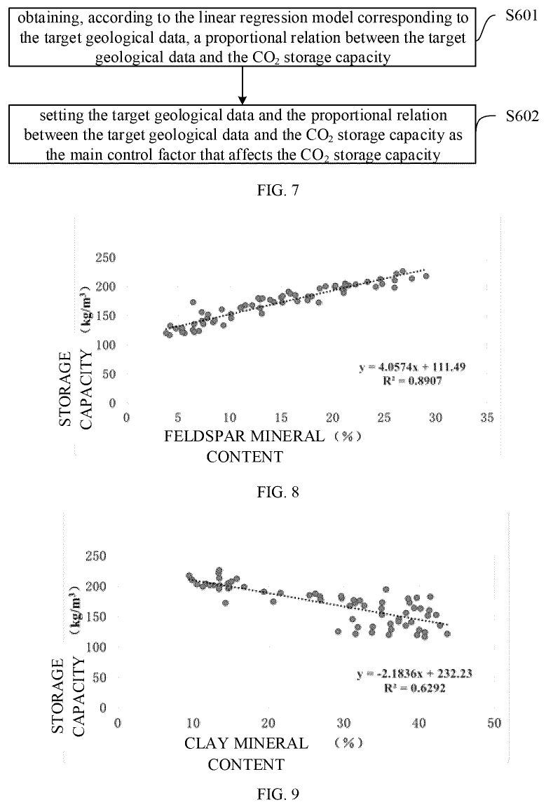

Further, with reference to FIG. 7, obtaining, according to the linear regression model corresponding to the target geological data, the main control factor that affects the CO₂ storage capacity further includes:
S601: obtaining, according to the linear regression model corresponding to the target geological data, a proportional relation between the target geological data and the CO₂ storage capacity; and
S602: setting the target geological data and the proportional relation between the target geological data and the CO₂ storage capacity as the main control factor that affects the CO₂ storage capacity.

If the slope of the linear regression model corresponding to the target geological data is positive, the target geological data is proportional to the CO₂ storage capacity. If the slope of the linear regression model corresponding to the target geological data is negative, the target geological data is inversely proportional to the CO₂ storage capacity. The target geological data and the corresponding proportional relation are set as the main control factor that affects the CO₂ storage capacity, that is, there may be one or more main control factors, and each main control factor includes a piece of target geological data and a corresponding proportional relation.

Finally, formations at the different depths in the study area are analyzed based on the main control factor, and a formation affected by the main control factor is determined as a formation with CO₂ storage potential.

The specific steps include:
step 2.1: acquiring target geological data at different depths for any location in the study area; and
step 2.2: integrating the target geological data at the plurality of different depths, selecting a target formation based on a proportional relation between the target geological data and the CO₂ storage capacity, and determining the target formation as a formation with CO₂ storage potential at any location in the study area.

Based on this, a formation with CO₂ storage potential at any location in the study area can be obtained, and then formations with CO₂ storage potential in the entire study area can be obtained.

If the target geological data is proportional to the CO₂ storage capacity (the larger the target geological data, the larger the CO₂ storage capacity), then the step 2.2 includes:
step 2.21: calculating an average value of the target geological data at all the different depths, determining the target geological data greater than the average value as pre-selected geological data, and setting a depth position corresponding to the pre-selected geological data as a pre-selected depth position;
step 2.22: determining whether there are other pre-selected depth positions within a set depth range above any one of the pre-selected depth positions,
if so, setting the pre-selected depth position and said other pre-selected depth positions as candidate depth positions, and
if not, setting the pre-selected depth position as a candidate depth position; and
step 2.23: determining whether there are other pre-selected depth positions within a set depth range below the pre-selected depth position,
if so, setting the pre-selected depth position and said other pre-selected depth positions as candidate depth positions, and
if not, setting the pre-selected depth position as a candidate depth position.

In this way, several candidate depth positions can be obtained, and it is necessary to further determine whether a depth difference between any two adjacent candidate depth positions is within the set depth range from top to bottom along the depth direction. If so, the two adjacent candidate depth positions are determined as the same candidate formation, and if not, the two adjacent candidate depth positions are determined as different candidate formations. The set depth range can be set according to actual conditions, which is not limited by the embodiment of the present disclosure.

If the target geological data is inversely proportional to the CO₂ storage capacity (the larger the target geological data, the smaller the CO₂ storage capacity), the step 2.2 includes a step 2.21: calculating an average value of the target geological data at all the different depths, determining the target geological data less than the average value as pre-selected geological data, and setting a depth position corresponding to the pre-selected geological data as a pre-selected depth position. The remaining steps and methods are similar, and will not be described in detail in the embodiment of the present disclosure.

In this way, one or more candidate formations can be obtained, and the uppermost candidate depth position and lowermost candidate depth position in the candidate formation are respectively used as upper limit and lower limit of the candidate formation. The depth range of the candidate formation is obtained according to a depth difference between the upper limit and lower limit. When there is only one candidate formation after analysis based on the target geological data, the candidate formation is determined as a target formation obtained based on the target geological data. When there are a plurality of candidate formations after analysis based on the target geological data, a candidate formation corresponding to the maximum depth range may be selected as the target formation obtained based on the target geological data.

The target geological data may be one or more. If there is one piece of target geological data, the target formation corresponding to the target geological data is the formation with CO₂ storage potential. If there are a plurality pieces of target geological data, the target formations corresponding to all of the target geological data are integrated to obtain a formation with CO₂ storage potential.

In addition to the above method for determining a formation with CO₂ storage potential, other methods can also be used to determine a formation with CO₂ storage potential. For example, the depth with the greatest CO₂ storage potential in a study area can be determined first, and then a formation with CO₂ storage potential can be further determined.

In a specific example, for the actual stratigraphic conditions of the Shahezi Formation in the Lishu Fault Depression of the Songliao Basin, a plurality pieces of geological data, such as temperature, pressure, mineral composition, etc., are considered, and corresponding models are established according to different depths. A simulation tool is used to simulate and calculate a plurality pieces of geological data at different depths of the Shahezi Formation to obtain CO₂ storage capacities at a plurality of different depths, with a total of 68 simulations.

Fitting was performed according to the calculation results of the storage capacity, and R² was calculated. According to the linear regression fitting of the contents of feldspar, clay minerals, quartz, and carbonates, R²>0.55, and main control factors are obtained. The feldspar mineral content and the storage capacity had the best fitting effect, with R² of 0.8907, followed by quartz, clay minerals, and carbonate minerals, which are 0.644, 0.6292, and 0.5995, respectively.

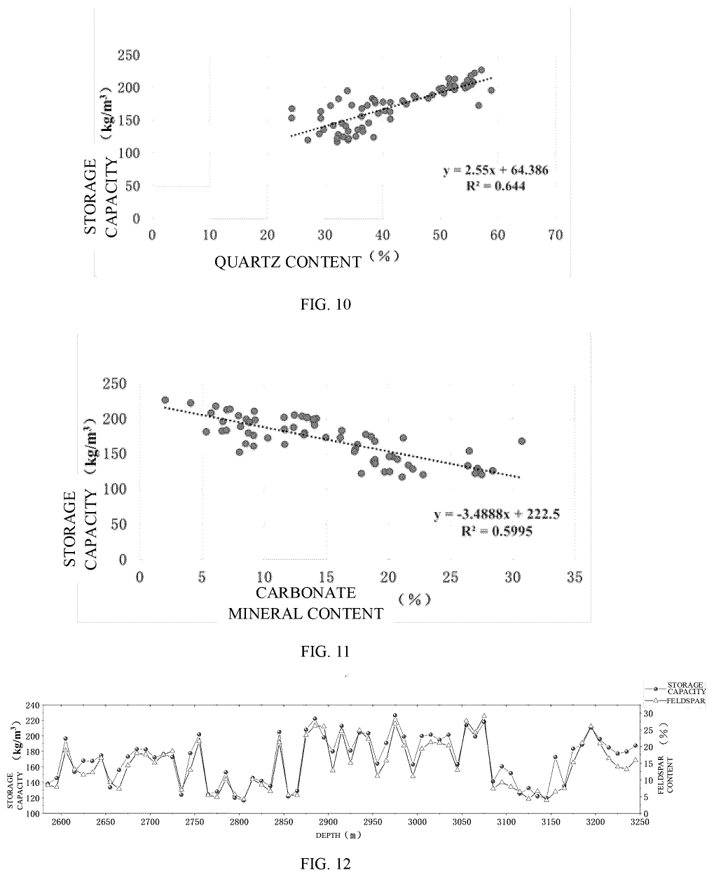

As shown in FIGS. 8 to 11, the increase in the contents of feldspar and quartz leads to an increase in the CO₂ storage capacity, and the increase in the contents of clay minerals and carbonates leads to a decrease in the CO₂ storage capacity. The order of affection on the CO₂ geological storage in the Shahezi Formation is feldspar>quartz>clay minerals>carbonate minerals, that is, higher contents of feldspar and quartz and lower contents of clay minerals and carbonate minerals are most suitable for CO₂ storage in the study area.

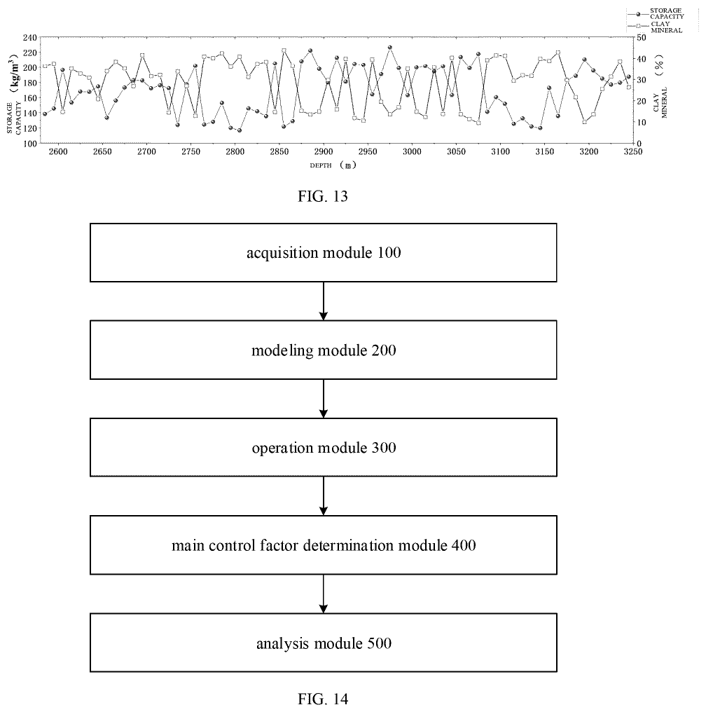

As shown in FIGS. 12 and 13, the main control factor is analyzed, and it is determined that the depth with the highest feldspar content and the lowest clay mineral content at different depths for a location in the study area is 2,975 m, with the greatest storage potential. In the upper sub-member of the second member of the Shahezi Formation, the formation from 2,875 m to 3,075 m is the formation with CO₂ storage potential, and the storage potential can reach 163 kg/m³ to 226.4 kg/m³.

It should be noted that the user information (including but not limited to user device information, user personal information, etc.) and data (including but not limited to data used for analysis, stored data, displayed data, etc.) involved in the present disclosure are all authorized by users or fully authorized by all parties. Moreover, the collection, storage, use, processing, transmission, provision, disclosure and application of relevant data comply with the relevant laws, regulations and standards of relevant countries and regions and do not violate public order and good customs, with necessary confidentiality measures taken, and provide corresponding operation portals for users to choose to authorize or refuse.

The present application provides users with corresponding big data analysis (such as personal biometrics, identity data, consumption data, asset data, electronic terminal operation data, etc.) operation portals for users to choose to agree or reject automated decision-making results. If a user chooses to reject, the expert decision-making process will be entered.

Based on the above method for evaluating CO₂ storage potential according to formation heterogeneity, the embodiment of the present disclosure also provides an apparatus for evaluating CO₂ storage potential according to formation heterogeneity. The apparatus may include a system (including a distributed system), software (application), module, component, server, client, etc. which use the method described in the embodiment of the present disclosure and may be combined with necessary implementation hardware. Based on the same innovative concept, the apparatus in one or more embodiments provided in the embodiment of the present disclosure is as described in the following embodiment. Since the implementation solution of the apparatus to solve the problem is similar to the method, the implementation of the specific apparatus in the embodiment of the present disclosure can refer to the implementation of the above method, and the repeated parts will not be repeated. The term “unit” or “module” used below can be a combination of software and/or hardware that implements the predetermined function. Although the apparatus described in the following embodiment is exemplarily implemented in software, the implementation in hardware, or a combination of software and hardware, is also possible and conceivable.

Specifically, FIG. 14 shows a schematic diagram of a module structure of an embodiment of the apparatus for evaluating CO₂ storage potential under a heterogeneous geological condition according to the embodiment of the present disclosure. As shown in FIG. 14, the apparatus for evaluating CO₂ storage potential according to formation heterogeneity according to the embodiment of the present disclosure includes an acquisition module 100, a modeling module 200, an operation module 300, a main control factor determination module 400, and an analysis module 500.

The acquisition module 100 is configured to acquire a plurality pieces of geological data at different depths for any location in a study area.

The modeling module 200 is configured to perform heterogeneity modeling according to the plurality pieces of geological data at each of the depths and CO₂ injection conditions, to obtain one-dimensional grid models respectively corresponding to a plurality of the different depths, and the CO₂ injection conditions of the one-dimensional grid models respectively corresponding to the plurality of different depths are the same.

The operation module 300 is configured to obtain, based on the one-dimensional grid models respectively corresponding to the plurality of different depths, CO₂ storage capacities at the plurality of different depths through simulation.

The main control factor determination module 400 is configured to integrate the CO₂ storage capacities at the plurality of different depths and fit the plurality pieces of geological data, to obtain a main control factor that affects the CO₂ storage capacity.

The analysis module 500 is configured to analyze formations at the different depths in the study area based on the main control factor, and determine a formation affected by the main control factor as a formation with CO₂ storage potential.

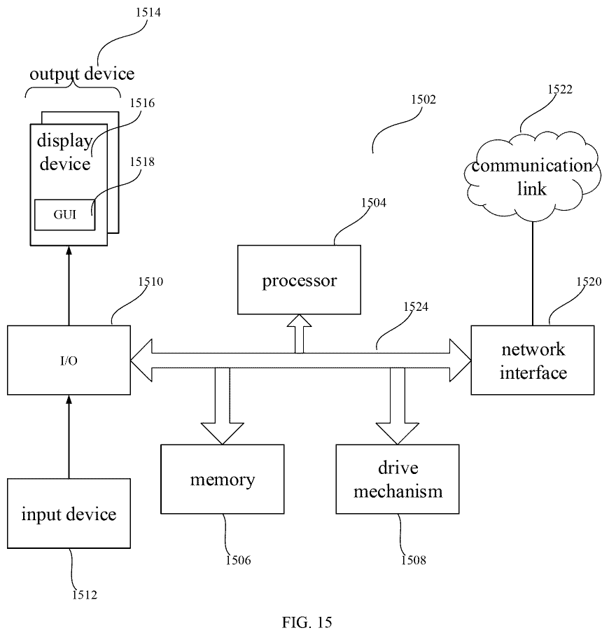

As shown in FIG. 15, an embodiment of the present disclosure further provides a computer device 1502, based on the above method for evaluating CO₂ storage potential according to formation heterogeneity, and the above method is executed on the computer device 1502. The computer device 1502 may include one or more processors 1504, such as one or more central processing units (CPUs) or graphics processing units (GPUs), and each processing unit may implement one or more hardware threads. The computer device 1502 may also include any memory 1506, which stores any kind of information such as code, settings, data, etc. In a specific embodiment, the memory 1506 stores a computer program that can be executed on the processor 1504. When the computer program is executed by the processor 1504, an instruction according to the above method can be executed. Without limitation, for example, the memory 1506 may include any one or more combinations of the following: any type of RAM, any type of ROM, flash memory device, hard disk, optical disk, etc. More generally, any memory may use any technology to store information. Further, any memory may provide volatile or non-volatile retention of information. Further, any memory may represent a fixed or removable component of the computer device 1502. In one embodiment, when the processor 1504 executes an associated instruction stored in any memory or combination of memories, the computer device 1502 may perform any operation of the associated instruction. The computer device 1502 also includes one or more drive mechanisms 1508 for interacting with any memory, such as a hard disk drive mechanism, an optical disk drive mechanism, etc.

The computer device 1502 may also include an input and output module 1510 (I/O) for receiving various inputs (via an input device 1512) and for providing various outputs (via an output device 1514). A specific output mechanism may include a display device 1516 and an associated graphical user interface 1518 (GUI). In other embodiments, the input and output module 1510 (I/O), the input device 1512, and the output device 1514 may not be included, and the computer device 1502 may be only used as a computer device in a network. The computer device 1502 may also include one or more network interfaces 1520 for exchanging data with other devices via one or more communication links 1522. One or more communication buses 1524 couple the components described above together.

The communication links 1522 may be implemented in any manner, for example, through a local area network, a wide area network (e.g., the Internet), a point-to-point connection, etc., or any combination thereof. The communication links 1522 may include any combination of hardwired links, wireless links, routers, gateway functions, name servers, etc. governed by any protocol or combination of protocols.

Corresponding to the method in FIGS. 1 to 7, the embodiment of the present disclosure further provides a computer-readable storage medium, on which a computer program is stored, and when being executed by the processor, the computer program implements the steps of the above method.

The embodiment of the present disclosure also provides a computer-readable instruction. When a processor executes the instruction, the program therein causes the processor to execute the method shown in FIGS. 1 to 7.

The embodiment of the present disclosure also provides a computer program product, and when being executed by the computer device, the computer program product implements the method shown in FIGS. 1 to 7.

The computer program product described in the present disclosure is a software product that mainly implements the method described in the present disclosure through a computer program.

It should be understood that in the various embodiments of the present disclosure, the serial numbers of the above processes do not mean the order of execution. The execution order of the processes should be determined by its function and internal logic, and should not constitute any limitation on the implementation process of the embodiments of the present disclosure.

It should also be understood that in the embodiments of the present disclosure, the term “and/or” is only a description of a correlation of associated objects, indicating that three relations may exist. For example, A and/or B may represent the existence of A alone, the existence of A and B at the same time, and the existence of B alone. In addition, the character “/” in the embodiments of the present disclosure generally indicates that the associated objects before and after “/” are in an “or” relation.

A person skilled in the art could recognize that the units and algorithm steps of the examples described in the embodiments disclosed in the embodiments of the present disclosure may be implemented by electronic hardware, computer software, or a combination of electronic hardware and computer software. In order to clearly illustrate the interchangeability of hardware and software, the composition and steps of the embodiments have been generally described in terms of function in the above description. Whether these functions are performed in hardware or software depends on the specific application and design constraints of the technical solution. Professional technicians could use different methods to implement the described functions for each specific application, but such implementation should not be considered to be beyond the scope of the embodiments of the present disclosure.

A person skilled in the art could clearly understand that, for the convenience and brevity of description, the specific working processes of the systems, devices and units described above may refer to the corresponding processes in the method embodiments described above and will not be repeated here.

In the several embodiments according to the present disclosure, it should be understood that the disclosed systems, apparatuses and methods may be implemented in other ways. For example, the apparatus embodiments described above are only schematic. For example, the division of the units is merely a logical function division, and it may be other division methods in actual implementation, such as multiple units or components may be combined or integrated into another system, or some features may be ignored or not executed. In addition, the mutual coupling or direct coupling or communication connection shown or discussed may be indirect coupling or communication connection through some interfaces, devices or units, or may be electrical, mechanical or other form of connection.

The units described as separate components may or may not be physically separated, and the components shown as units may or may not be physical units, that is, they may be located in one place or distributed on multiple network units. Some or all of the units may be selected according to actual needs to achieve the purpose of the embodiments of the present disclosure.

In addition, the functional units in the embodiments of the present disclosure may be integrated into one processing unit, or the units may exist physically separately, or two or more units may be integrated into one unit. The above integrated unit may be implemented in the form of hardware or in the form of software functional units.

If the integrated unit is implemented in the form of a software functional unit and sold or used as an independent product, the integrated unit can be stored in a computer-readable storage medium. Based on such understanding, the technical solutions of the embodiments of the present disclosure can essentially be embodied in the form of a software product, or the part that contributes to the related art, or all or part of the technical solutions can be embodied in the form of a software product. The computer software product is stored in a storage medium and includes several instructions for a computer device (which can be a personal computer, server, or network device, etc.) to perform all or part of the steps of the method described in the embodiments of the present disclosure. The above storage medium includes various media that can store program codes, such as USB flash drives, mobile hard disks, read-only memories (ROM), random access memories (RAM), disks or optical disks.

Specific embodiments are used in the present disclosure to illustrate the principles and implementation methods of the embodiments of the present disclosure. The description of the above embodiments is merely used to help understand the methods and core ideas of the embodiments of the present disclosure. At the same time, a person skilled in the art may make changes in the specific implementation methods and application scopes based on the ideas of the embodiments of the present disclosure. In summary, the content of the present disclosure should not be understood as a limitation on the embodiments of the present disclosure.

1. A method for evaluating CO₂ storage potential under a heterogeneous geological condition, comprising:
acquiring a plurality pieces of geological data at different depths for any location in a study area;
performing heterogeneity modeling according to the plurality pieces of geological data at each of the depths and CO₂ injection conditions, to obtain one-dimensional grid models respectively corresponding to a plurality of the different depths, wherein the CO₂ injection conditions of the one-dimensional grid models respectively corresponding to the plurality of different depths are the same;
obtaining, based on the one-dimensional grid models respectively corresponding to the plurality of different depths, CO₂ storage capacities at the plurality of different depths through simulation;
integrating the CO₂ storage capacities at the plurality of different depths and fitting the plurality pieces of geological data, to obtain a main control factor that affects the CO₂ storage capacity; and
analyzing formations at the different depths in the study area based on the main control factor, and determining a formation affected by the main control factor as a formation with CO₂ storage potential.

2. The method according to claim 1, wherein the plurality pieces of geological data comprise logging data and formation water data.

3. The method according to claim 1, wherein the performing heterogeneity modeling according to the plurality pieces of geological data at each of the depths and the CO₂ injection conditions, to obtain one-dimensional grid models respectively corresponding to the plurality of the different depths further comprises:
preprocessing the plurality pieces of geological data at each of the depths to obtain a plurality pieces of preprocessed geological data at each of the depths, and
simulating formation characteristics at each of the depths using a simulation tool, according to the plurality pieces of preprocessed geological data at each of the depths and the CO₂ injection conditions, to obtain one-dimensional grid models respectively corresponding the plurality of different depths.

4. The method according to claim 1, wherein the integrating the CO₂ storage capacities at the plurality of different depths and fitting the plurality pieces of geological data, to obtain the main control factor that affects the CO₂ storage capacities further comprises:
integrating the CO₂ storage capacities at the plurality of different depths and the same geological data at the plurality of different depths, to obtain correlations between the CO₂ storage capacity and each geological data in the study area, and
analyzing, according to the correlations, a degree of affection of each geological data on the CO₂ storage capacity, to obtain the main control factor that affects the CO₂ storage capacity.

5. The method according to claim 4, wherein the integrating the CO₂ storage capacities at the plurality of different depths and the same geological data at the plurality of different depths, to obtain the correlations between the CO₂ storage capacity and each geological data in the study area further comprises:
constructing scatter plots corresponding to the geological data using the CO₂ storage capacities at the plurality of different depths and the same geological data at the plurality of different depths as a plurality of groups of ordinate values and abscissa values, respectively, and
obtaining, based on the scatter plots corresponding to each geological data, correlations between the CO₂ storage capacity and each geological data in the study area.

6. The method according to claim 4, wherein the analyzing, according to the correlations, the degree of affection of each geological data on the CO₂ storage capacity, to obtain the main control factor that affects the CO₂ storage capacity further comprises:
obtaining, according to the scatter plots corresponding to each geological data, linear regression models corresponding to each geological data,
calculating, according to the linear regression model, goodness-of-fit values corresponding to each geological data,
setting geological data corresponding to a goodness-of-fit value greater than a set value or a maximum value among all goodness-of-fit values as target geological data, and
obtaining, according to the linear regression model corresponding to the target geological data, the main control factor that affects the CO₂ storage capacity.

7. The method according to claim 6, wherein the obtaining, according to the linear regression model corresponding to the target geological data, the main control factor that affects the CO₂ storage capacity further comprises:
obtaining, according to the linear regression model corresponding to the target geological data, a proportional relation between the target geological data and the CO₂ storage capacity, and
setting the target geological data and the proportional relation between the target geological data and the CO₂ storage capacity as the main control factor that affects the CO₂ storage capacity.

8. An apparatus for evaluating CO₂ storage potential under a heterogeneous geological condition, comprising:
an acquisition module configured to acquire a plurality pieces of geological data at different depths for any location in a study area;
a modeling module configured to perform heterogeneity modeling according to the plurality pieces of geological data at each of the depths and CO₂ injection conditions, to obtain one-dimensional grid models respectively corresponding to a plurality of the different depths, wherein the CO₂ injection conditions of the one-dimensional grid models respectively corresponding to the plurality of different depths are the same;
an operation module configured to obtain, based on the one-dimensional grid models respectively corresponding to the plurality of different depths, CO₂ storage capacities at the plurality of different depths through simulation;
a main control factor determination module configured to integrate the CO₂ storage capacities at the plurality of different depths and fit the plurality pieces of geological data, to obtain a main control factor that affects the CO₂ storage capacity; and
an analysis module configured to analyze formations at the different depths in the study area based on the main control factor, and determine a formation affected by the main control factor as a formation with CO₂ storage potential.

9. A computer device, comprising: a memory; a processor; and a computer program stored in the memory, wherein when being executed by the processor, the computer program implements an instruction of a method, wherein the method comprises:
acquiring a plurality pieces of geological data at different depths for any location in a study area;
performing heterogeneity modeling according to the plurality pieces of geological data at each of the depths and CO₂ injection conditions, to obtain one-dimensional grid models respectively corresponding to a plurality of the different depths, wherein the CO₂ injection conditions of the one-dimensional grid models respectively corresponding to the plurality of different depths are the same;
obtaining, based on the one-dimensional grid models respectively corresponding to the plurality of different depths, CO₂ storage capacities at the plurality of different depths through simulation;
integrating the CO₂ storage capacities at the plurality of different depths and fitting the plurality pieces of geological data, to obtain a main control factor that affects the CO₂ storage capacity; and
analyzing formations at the different depths in the study area based on the main control factor, and determining a formation affected by the main control factor as a formation with CO₂ storage potential.

10. The computer device according to claim 9, wherein the plurality pieces of geological data comprise logging data and formation water data.

11. The computer device according to claim 9, wherein the performing heterogeneity modeling according to the plurality pieces of geological data at each of the depths and the CO₂ injection conditions, to obtain one-dimensional grid models respectively corresponding to the plurality of the different depths further comprises:
preprocessing the plurality pieces of geological data at each of the depths to obtain a plurality pieces of preprocessed geological data at each of the depths, and
simulating formation characteristics at each of the depths using a simulation tool, according to the plurality pieces of preprocessed geological data at each of the depths and the CO₂ injection conditions, to obtain one-dimensional grid models respectively corresponding the plurality of different depths.

12. The method according to claim 9, wherein the integrating the CO₂ storage capacities at the plurality of different depths and fitting the plurality pieces of geological data, to obtain the main control factor that affects the CO₂ storage capacities further comprises:
integrating the CO₂ storage capacities at the plurality of different depths and the same geological data at the plurality of different depths, to obtain correlations between the CO₂ storage capacity and each geological data in the study area, and
analyzing, according to the correlations, a degree of affection of each geological data on the CO₂ storage capacity, to obtain the main control factor that affects the CO₂ storage capacity.

13. The method according to claim 12, wherein the integrating the CO₂ storage capacities at the plurality of different depths and the same geological data at the plurality of different depths, to obtain the correlations between the CO₂ storage capacity and each geological data in the study area further comprises:
constructing scatter plots corresponding to the geological data using the CO₂ storage capacities at the plurality of different depths and the same geological data at the plurality of different depths as a plurality of groups of ordinate values and abscissa values, respectively, and
obtaining, based on the scatter plots corresponding to each geological data, correlations between the CO₂ storage capacity and each geological data in the study area.

14. The method according to claim 12, wherein the analyzing, according to the correlations, the degree of affection of each geological data on the CO₂ storage capacity, to obtain the main control factor that affects the CO₂ storage capacity further comprises:
obtaining, according to the scatter plots corresponding to each geological data, linear regression models corresponding to each geological data,
calculating, according to the linear regression model, goodness-of-fit values corresponding to each geological data,
setting geological data corresponding to a goodness-of-fit value greater than a set value or a maximum value among all goodness-of-fit values as target geological data, and
obtaining, according to the linear regression model corresponding to the target geological data, the main control factor that affects the CO₂ storage capacity.

15. The method according to claim 14, wherein the obtaining, according to the linear regression model corresponding to the target geological data, the main control factor that affects the CO₂ storage capacity further comprises:
obtaining, according to the linear regression model corresponding to the target geological data, a proportional relation between the target geological data and the CO₂ storage capacity, and
setting the target geological data and the proportional relation between the target geological data and the CO₂ storage capacity as the main control factor that affects the CO₂ storage capacity.
